(19) United States
(12) Patent Application Publication
(10) Pub. No.: US 2004/0071592 A1
(43) Pub. Date: Apr. 15, 2004

(54) Fast dissolving solid ortho-phthalic aldehyde formulations

(76) Inventor: Ioana Annis, Mundelein, IL (US)

Correspondence Address: THE DOW CHEMICAL COMPANY, INTELLECTUAL PROPERTY SECTION, P. O. BOX 1967, MIDLAND, MI, 48641-1967, US

(21) Appl. No.: 10/269,168

(22) Filed: Oct. 10, 2002

(51) Int. Cl. A01N 1/00; A61L 2/00

(52) U.S. Cl. 422/36; 422/28; 422/131

(57) ABSTRACT

The invention comprises a solidified melt mixture of OPA with compounds selected from the group of water soluble polymers, surfactants, carboxylic acids, and salts wherein the formulation contains from 0.1% to 99.9% OPA, better 10% to 50% OPA, best about 20 to 30% OPA.

### BACKGROUND OF THE INVENTION

1. Field of the Invention

The invention comprises fast-dissolving solid ortho-phthalic aldehyde (OPA) compositions. In particular, the invention is directed towards solid melt mixtures of OPA that are quickly dissolvable in water and other solvents.

2. Description of the Prior Art

Ortho-Phthalic aldehyde (OPA) is gaining increasing recognition in the market place due to its broad spectrum biocidal efficacy and its low odor. One of the challenges in using solid OPA is its slow dissolution in water, even at low concentrations. Most often, an organic solvent must be employed as a solubility aid. Thus, OPA is currently offered in the biocides market as prepared low concentration aqueous solutions (usually comprising 0.55% OPA). In a variety of markets, a solid formulation of the biocide would be desirable based on the convenience of handling a smaller volume of a concentrated solid versus a large volume of dilute liquid.

The solid concentrated formulation would be advantageous for several reasons.

1) A solid product reduces potential for hazardous spills since any “spilled” product can be readily swept up and disposed of, whereas a liquid product will soak into permeable surfaces (carpets, wood, under tiles, grout, etc.) making complete removal difficult. Furthermore, cleaning up the bulk of the spilled liquid with absorbents or spill mats creates a much larger amount of waste to be disposed.

2) The amount of storage space required for a concentrate versus a dilute formulation would be less which is an advantage to customers.

3) Disposal of packaging materials is also reduced for a concentrated solid product compared to a dilute liquid product.

4) The storage stability of solid products is typically better than dilute solutions. In the commercially available liquid product, air can oxidize the active ingredient (a dialdehyde) which decreases the activity of the product during storage. Air oxidation is significantly less in solid products due to reduction in the area of contact between air and the active ingredient (primarily due to diffusion limitations of the air into the solid). Furthermore, a concentrated product has significantly less surface area of packaging exposed to air compared to a dilute product. Reducing the packaging surface area reduces the infiltration of air into the packaged product and thereby increases product life (given similar packaging materials).

5) The safety in handling a product, which should not come into direct contact with skin or eyes, is improved with a low dusting solid product compared to a liquid product. Liquid products can splash while pouring which often requires eye shielding (safety glasses or goggles or equivalent). A low dusting solid product would greatly mitigate the potential for eye exposure.

OPA is typically used in direct applications such as instrument sterilization. As an example, 0.55% and 5% aqueous solutions of OPA are currently on the market. These solutions are most often used in automated endoscope reprocessors, which are used to disinfect endoscopes.

Desirably, a composition would exist that could be quickly dissolved into water or another solvent and then applied as a biocidal solution for various applications.

### SUMMARY OF THE INVENTION

The invention comprises a melt mixture of OPA with one or more compounds selected from the group consisting of water soluble polymers, carboxylic acids, surfactants, and salts. OPA is present in loadings in the range of 0.1 to 99.9%, preferably 10 to 50%, and most preferably 20 to 30%.

### DETAILED DESCRIPTION OF PREFERRED EMBODIMENTS

The invention comprises solid formulations of OPA which are quickly dissolvable in water and other solvents through the addition of various additives to the OPA. Melt mixtures of OPA with water soluble polymers, carboxylic acids, surfactants, and salts at about 25% loading of OPA, have dissolution times up to 15 times faster than granular OPA, and up to 45 times faster than caked OPA. The term water soluble polymer as used herein, indicates that the polymer has a water solubility of at least 0.5 g in 100 g of water. Suitable polymers include polyethylene glycol of molecular weights from 1000 to 8,000,000, polyvinyl alcohol, polyvinyl acetate, homo and copolymers of maleic acid, polyacrylic acid, polyacrylates, polyacrylamide and polyacrylamide derivatives, poly(amino acids) such as polyaspartic acid, polycarbonates, saccharides and polysaccharides, cellulose and cellulose derivatives, cyclodextrin and cyclodextrin derivatives, and starches.

Carboxylic acids as used herein are compounds containing at least one carboxylic functionality (COOH). Other functionalities could be present in the molecule, and could improve the dissolution efficiency. However, when choosing the additives, the compatibility with the aldehyde functionality should be considered. Non-limiting examples of useful carboxylic acids are citric acid, tartaric acid, glutaric acid, malonic acid, adipic acid, succinic acid, and oxalic acid.

Surfactants are well known to improve biocidal efficiency. In the context of this invention, surfactants could have a dual role of dissolution enhancers and efficiency enhancers. The surfactant of choice in the context of this invention could be anionic, cationic, amphoteric, or nonionic. Anionic surfactants of particular interest are sodium dodecyl sulfate and dinonylsulfoccinate. Cationic surfactants of interest are the quaternary ammonium compounds which alone could exhibit significant cidal efficiency.

Salts as used herein, refer to organic and inorganic compounds comprising an ionic or partial ionic interaction between a positive (or partial positive) charged component and a negative (or partial negative) charged component. Of particular usefulness to this invention are the carboxylic acid salts. Carboxylic acid salts are defined as derivatives of carboxylic acids where the proton from the carboxyl functionality has been substracted, and the negative charge on the carboxy functionality is balanced by a positive charged ion, such as a metal ion, or a positive charged organic ion. Non-limiting examples of carboxylic acid salts are sodium citrate, sodium tartrate, sodium acetate, sodium acetate trihydrate, and potassium acetate. Those skilled in art would appreciate that some of these salts are known disintegrating agents. Other non limiting examples of salts pertinent to this invention include chlorides such as sodium chloride; phosphates such as sodium phosphate, potassium phosphate, potassium hydrogen phosphate; carbonates such as sodium carbonate and potassium carbonate; sulfates such as sodium sulfate; sulfoxylates, such as sodium sulfoxylate and sodium formaldehyde sulfoxylate, etc.

As will be appreciated by those skilled in the art, dissolution agents employed may also improve the cleaning efficiency of the final disinfectant solution. Additives such as buffering salts, chelating agents, stabilizers, disintegrating agents, organic matter dispersants, antiscaling agents, and additional biocides may be added to the final formulation for further improvement of disinfectant properties. Advantageously, such an approach would overcome the challenges encountered when using pure OPA, such as severe caking and hardening, and the subsequent need to use an organic cosolvent.

The pH buffering salts could be added to the solid composition to ensure that after dissolution the pH of the antimicrobial solution is in the desired pH range. Any buffer that can achieve a desired pH range can be used. However, it should be noted that aldehydes are incompatible with buffering systems containing primary or secondary amines. Dialdehydes especially react to crosslink primary or secondary amines resulting in reduction in the concentration of both aldehyde and buffering salt. It should also be noted that the antimicrobial efficiency of OPA is increased in slightly alkaline media, however there are applications where a pH outside this range is preferred.

The chelating agents as used herein, are defined as molecules comprising nonmetal atoms, two or more of which atoms are capable of linking or binding with a metal ion to form a heterocyclic ring including the metal ion. Preferable chelators for use in the present invention include, but are not limited to, ethylenediamine-N,N,N′,N′-tetraacetic acid (EDTA); the disodium, trisodium, tetrasodium, dipotassium, tripotassium, dilithium and diammonium salts of EDTA; citric acid, trans-1,2-diaminocyclohexane-N,N,N′,N′-tetraacetic acid monohydrate; N,N-bis(2-hydroxyethyl)glycine; 1,3-diamino-2-hydroxypropane-N,N,N′,N′-tetraacetic acid; 1,3-diaminopropane-N,N,N′,N′-tetraacetic acid; ethylenediamine-N,N′-diacetic acid; ethylenediamine-N,N′-dipropionic acid dihydrochloride; ethylenediamine-N,N′-bis(methylenephosphonic acid) hemihydrate; N-(2-hydroxyethyl)ethylenediamine-N,N′,N′-triacetic acid; ethylenediamine-N,N,N′,N′-tetrakis(methylenephosponic acid); O,O′-bis(2-aminoethyl)ethyleneglycol-N,N,N′,N′-tetraacetic acid; N,N-bis(2-hydroxybenzyl)ethylenediamine-N,N-diacetic acid; 1,6-hexamethylenediamine-N,N,N′,N′-tetraacetic acid; N-(2-hydroxyethyl)iminodiacetic acid; iminodiacetic acid; 1,2-diaminopropane-N,N,N′,N′-tetraacetic acid; nitrilotriacetic acid; nitrilotripropionic acid; the trisodium salt of nitrilotris(methylenephosphoric acid); 7,19,30-trioxa-1,4,10,13,16,22,27,33-octaazabicyclo[11,11,11]pentatriacontane hexahydrobromide; and triethylenetetramine-N,N,N′,N″,N′″,N′″-hexaacetic acid.

Stabilizers, as defined for the purpose of this invention, are additives included in the formulation to improve the stability of the solid mix, the stability of the disinfectant solution prepared by dissolving the solid formulation in a suitable solvent system, or additives to improve the compatibility of all the reagents in solid or liquid form. Non-limiting examples of stabilizers are: organic additives meant to increase the in-solution compatibility of the buffering salts with the organic matrix, such as water miscible alcohols, dicarboxylic acid esters, propylene carbonate, N,N-dimethylacetamide and butyrolactone; photostabilizers used to improve the light stability of OPA; and chelants used to reduce metal-induced oxidation of OPA.

Disintegrating agents are defined as compounds or mixtures of compounds that are added to a tablet or a solid formulation to facilitate its breakup or disintegration in suitable solvents. Water soluble disintegrating agents are preferred in certain applications of this invention, such as medical instrument disinfection. Other applications can accommodate a water insoluble disintegrating agent. Disintegrating agents, also known as dissolution enhancers or disintegrants, are well known to those skilled in the art. Nonlimiting examples are: carboxylates, such as potassium acetate, sodium acetate and sodium citrate; water soluble polymers, such as polyethylene glycols and polyacrylates, such as those marketed by Rohm and Haas under the name of Acusol®; starches; cellulose and wood products; cellulose derivatives; cation-exchange resins such as Amberlite IRP-88; alginic acid; guar gum; citrus pulp; combinations of starch with surfactants; and crosslinked, swellable polymers.

Other active agents may include additional algicides, bactericides, antiviral compounds, fungicides, corrosion inhibitors, scale inhibitors, organic matter dispersants, complexing agents, surfactants, enzymes, biocides and other compatible products which will lend greater functionality to the product. These agents may be added to the solid composition at a dosage such that when dissolved to form an antimicrobial solution, the final dosage is known by those skilled in the art to be efficacious.

Other biocides that may be added to the solid formulation of this invention are organic nitrogen-sulfur-containing compounds, halogenated organic compounds, nitrogen-containing organic compounds, sulfur-containing organic compounds and phenols.

Non-limiting examples of organic nitrogen-sulfur containing compounds are alkylene bisthiocyanates, such as methylene bisthiocyanate; 3-isothiazolone compounds, such as 5-chloro-2-methyl-4-isothiazolin-3-one, 2-methyl-4-isothiazolin-3-one, 4,5-dichloro-2-n-octylisothiazolin-3-one, 1,2-benzoisothiazolin-3-one, 2-n-octylisothiazolin-3-one, etc.; dithiocarbamates, such as ammonium N-methylthiocarbamate, sodium N-methylthiocarbamate, dimethyldithiocarbamate (sodium salt), ethylene thiuram monosulfide, disodium_ethylenebisdithiocarbamate, manganese ethylenebisdithiocarbamate, etc.; sulfone amide compounds, such as chloramine T, N,N-dimethyl-N′-(fluorodichloromethylthio)-N′-phenylsulfamide, etc.; thiazole compounds, such as 2-(4-thiocyanomethylthio)benzothiazole, 2-mercaptobenzothiazole sodium, etc.; triazine compounds, such as hexahydro-1,3,5-tris(2-ethyl)-s-triazine, hexahydro-1,3,5-tris(2-hydroxyethyl)-s-triazene, etc.; and N-(fluorodichloromethylthio) phthalimide, 3,5-dimethyl-1,3,5-2H-tetrahydrothiadiazie-2-thione, dithio-2,2′-bis(benzomethylamide), etc.

Non-limiting examples of halogenated organic compounds for use with the solid formulation may include: organic halogenated cyano compounds, such as 2,2-dibromo-3-nitrilopropionamide and 2-bromo-2-bromomethylglutaronitrile; organic halogenated nitro compounds, such as 2-bromo-2-nitropropane-1,3-diol, 2,2-dibromo-2-nitroethanol, 1,1-dibromo-1-nitro-2-propanol, 1,1-dibromo-1-nitro-2-acetoxyethane, 1,1-dibromo-1-nitro-2-acetoxypropane, 2-bromo-2-nitro-1,3-diacetoxypropane, tribromonitromethane, b-bromo-b-nitrostyrene, 5-bromo-5-nitro-1,3-dioxane, 5-bromo-2-methyl-5-nitro-1,3-dioxane, etc.; organic bromoacetic acid esters or amides, such as 1,2-bis(bromoacetoxy)ethane, 1,2-bis(bromoacetoxy)propane, 1,4-bis(bromoacetoxy)-2-butene, methylene bisbromoacetate, benzyl bromoacetate, N-bromoacetamide, 2-bromoacetamide, etc.; and halogenated oxime compounds, such as 2-bromo-4′-hydroxyacetophenone, α-bromocinnamaldehyde, bistribromomethylsulfone, hexabromodimethylsulfone, 2-hydroxyethyl-2,3-dibromopropionate, 3-acetoxy-1,1,2-triiodo-1-propane, triiodoallyl alcohol, dichloroglyoxime, α-chlorobenzaldoxime acetate, and α-chlorobenzaldoxime; chloroisocyanurates; chlorhexidine; and halogen containing hydantoins.

The nitrogen containing organic compounds that could be used with this formulation may include, dodecyl guanidine hydrochloride, N-4-dihydroxy-α-oxobenzene ethane imidoyl chloride; chlorinated isocyanuric acid compounds, such as sodium dichloroisocyanurate, trichloroisocyanuric acid, etc.; quaternary ammonium compounds, such as dequalinium chloride, alkylisoquinolium bromides, benzalconium chloride, etc.; carbamic acid or its esters, such as methyl 2-benzoimidazolyl carbamate, 3-iodo-2-propargylbutyl carbamate, etc.; imidazole compounds, such as 1-[2-(2,4-dichlorophenyl)-2′-[(2,4-dichlorophenyl)methoxy]ethyl-3-(2-phenylethyl)-H-imid azolium chloride, 1-[2-(2,4-dichlorophenyl)-2-(2-propenyloxy)ethyl]-1H-imidazole, etc.; amide compounds, such as 2-(2-furyl)-3-(5-nitro-2-furyl)acrylic acid amide, 2-chloroacetamide, etc.; aminoalcohol compounds, such as N-(2-hydroxypropyl)aminomethanol, 2-(hydroxymethyl)aminoethanol, etc.; 2-pyridinethiol sodium oxide; N-(2-methyl-1-naphthyl) maleimide; and isophthalonitrile compounds, such as poly[oxyethylene(dimethylimino)ethylene(dimethylimino)ethylene dichloride], 5-chloro-2,4,6-trifluoroisophthalonitrile, 2,4,5,6-tetrachloroisophthalonitrile, etc.

The organic sulfur antibacterial ingredients used in the antibacterial agent for industrial use of this invention are not particularly limited; they may include, for example, 3,3,4,4-tetrachlorotetrahydrothiophene-1,1-dioxide, dithio-2,2′-bis-1-benzomethylamide, 2-hydroxypropylmethane thiosulfonate, ethylenethiurdam monosulfide, 4,5-dichloro-1,2-dithiol-3-one, hexabromodimethylsulfone, etc.

The phenolic compounds that have potential applications with this invention are not particularly limited; they may include, for example, 2,5-dichloro-4-bromophenol, 2,4,6-trichlorophenol, 2,4,6-tribromophenol, dichlorophene, cresol, resorcinol, ortho-phenyl phenol, etc.

Other potential additives include peroxy salts (salts which produce hydrogen peroxide in water), percarbonate, peracetate, persulfate, peroxide, or perborate salts, parabens, and silver sulfonamides.

The invention further comprises a method of sterilizing surgical instruments, comprising using the solid OPA of the invention as a high level disinfectant (“HLD”). As an example, the invention may comprise a single dose sealed tablet or a repeat use block which may then be used as the basis for a repeat use HLD or sterilization process, such as for an endoscope reprocessor. The solid would have significant convenience and safety benefits over current OPA applications, which are 45 times more dilute and a spill hazard. Further, the OPA of the invention may be tailored to specific applications through the careful selection of additives to, for example, improve the overall cleaning performance of the product by adding surfactants, and or organic and inorganic matter dispersants, and or an additional biocide.

A quick dissolving block, puck, pellet or powder offers several market opportunities including; HLD instrument sterilization, household and industrial cleaning, disinfection, and sanitization, industrial water treatment including cooling water, pulp and paper and oil field treatment, drilling mud preservative, sump treatment for metal working systems and a solid disinfectant for toilet bowls or porta-potties.

As used herein, the phrases “antimicrobial” and “inhibiting microbial growth” describe the killing of, as well as the inhibition of or control of, the growth of bacteria, viruses, yeasts, fungi, protozoa and algae. A number of important industries can experience serious adverse effects from the activity of such bacteria and fungi on the raw materials which they employ, on various aspects of their manufacturing activities, or on the finished products which they produce. Such industries include the paint, household and industrial cleaners, wood, textile, cosmetic, leather, tobacco, fur, rope, paper, pulp, plastics, fuel, oil, rubber, and machine industries.

Important applications of the composition of the present invention include: inhibiting the growth of bacteria and fungi in aqueous paints, adhesives, latex emulsions, and joint cements; preserving wood; preserving cutting oils; controlling slime-producing bacteria, fungi and protozoa in pulp and paper mills and cooling towers; as a spray or dip treatment for textiles and leather to prevent mold growth; as a component of anti-fouling paints to prevent adherence of fouling organisms; protecting paint films, especially exterior paints, from attack by fungi which occurs during weathering of the paint film; protecting processing equipment from slime deposits during manufacture of cane and beet sugar; preventing microorganism buildup and deposits in air washer or scrubber systems and in industrial fresh water supply systems; controlling microorganism contamination and deposits in oil field drilling fluids and muds, and in secondary petroleum recovery processes; preventing bacterial and fungal growth in paper coating processes which might adversely affect the quality of the paper coating; controlling bacterial and fungal growth and deposits during the manufacture of various specialty boards, e.g., cardboard and particle board; preventing sap stain discoloration on freshly cut wood of various kinds; controlling bacterial and fungal growth in clay and pigment slurries of various types which are manufactured for later use in paper coating and paint manufacturing for example, and which are susceptible to degradation by microorganisms during storage and transport; as a hard surface disinfectant to prevent growth of bacteria and fungi on walls, floors, etc.; and in swimming pools to prevent algae growth.

The control of bacteria and fungi in pulp and paper mill water systems which contain aqueous dispersions of papermaking fibers is especially important. The uncontrolled buildup of slime produced by the accumulation of bacteria and fungi causes offgrade production, decreased production due to breaks and greater cleanup frequency, increased raw material usage, and increased maintenance costs. The problem of slime deposits has been aggravated by the widespread use of closed white water systems in the paper industry.

Another important area where control of bacterial and fungal growth is vital is in clay and pigment slurries. These slurries are of various clays, e.g. kaolin, and pigments, e.g. calcium carbonate and titanium dioxide, and are manufactured usually at a location separate from the end use application, in for example, paper coating and paint manufacturing, and are then stored and held for later transport to the end use location. Because of the high quality standards for the paper and paint final products in which the slurry is used, it is essential that the clay or pigment slurry have a very low microorganism count or content so that it is usable in the paper coating or paint manufacturing.

The present invention discloses a family of readily soluble solid formulations of OPA. Thus, melt mixtures of OPA and one or more compounds selected from the group of water soluble polymers, surfactants, carboxylic acids, and salts, that were subsequently cooled and pulverized, showed significantly faster dissolution times in water as compared to OPA. The resulting solids had concentrations of from about 10% to about 67% OPA. As an example, a solid formulation containing 12.5% OPA and 87.5% polyethylene glycol dissolved 15 to 45 times faster than OPA alone to provide a 0.55% aqueous solution. As indicated above, this concentration is typical of that used in the instrument disinfection market.

Industrial processes producing a solid product having a high surface area per volume ratio are advantageous. High surface area per volume solids typically dissolve faster due to increased contact between the solid and the solubilizing liquid (typically water) in the end use application. Particle shape is relatively unimportant as long as the solvent (typically water) can intercalate between the particles upon submersion. Industrial processes which can yield suitable particles include, but are not limited to: drum flaking, conveyor flaking, pastillization, prilling, freeze spraying, jet precipitation or solidification, extrusion into strands or films, and pulverization/grinding/chopping.

Surface active agents may be added to aid wetting of the solid particle surfaces in aqueous applications. Anti-blocking or flow agents may also be added including inorganic salts, fumed silica, or precipitated silica to reduce the solid particles from blocking, cold flowing, or sticking together during transport and storage.

The OPA solids of the invention were prepared by various methods as determined by the physical properties of the additives employed. In the cases where the additives had a melting point under 100° C., the additive(s) were melted in a hot water bath at a temperature of about 90-100° C. followed by addition of ortho-phthalic aldehyde. The ratio of ortho-phthalic aldehyde to additive(s) was from about 1:10 to about 1:0.75. Heating of the mixture was continued along with gentle stirring until the OPA was completely melted. The melted mixture was then poured into a crystallizing dish and allowed to cool and solidify. The solid was removed and pulverized. In the cases where the additives had melting points above 100° C., OPA was heated in a hot water bath of temperature of about 80-90° C. until melted. The additive(s) were then added with stirring to ensure good mixing and coating of the additive particles with OPA. The mix was allowed to cool and solidify. The solid could be pulverized or dissolved as a block. Various compounds could be used according to the teaching of the invention. On an industrial scale, co-extrusion or melt mixing could be employed with equal efficacy.

### EXAMPLES

The following Examples 1-7 provide a brief overview of the various additives which may be used in the invention and the influence of each on the dissolution times of OPA. In all cases, the final solution was analyzed by High Performance Liquid Chromatography (“HPLC”) for OPA content and the presence of OPA in the targeted range was confirmed (0.45-0.55%). The formulations were prepared by either melting the OPA and the additive together followed by cooling, or for the high melting point additives, by melting the OPA and uniformly mixing in the additive. In both cases, the mix was allowed to cool and solidify. The solid could be flaked (described in the following Tables as melt mix/flakes), pulverized (described in the following Tables as melt mix/powder), or dissolved as a block (described in the following Tables as melt mix/block). Alternatively, but less efficient, is the dissolution in water of the OPA and the additives followed by lyophilization (freeze drying) to generate the solid mix. The freeze drying condition require close monitoring to prevent the sublimation of OPA. As a control, some solids were not melted but just simply mixed together as solids (described in the following Tables as physical mix). The results of these solid mixtures are shown in the control data.

In most cases solid formulations containing 25% OPA were prepared. This is an arbitrary concentration, selected for the purpose of demonstrating the superior solubility of the disclosed formulations, and is not meant to limit in any way the range of applicability of this patent. The dissolution time as reported herein, is defined as the time necessary to prepare aqueous OPA solutions containing 0.5±0.05% OPA. The dissolution was tested at room temperature, and in the presence of mechanical agitation. Upon visual determination of complete dissolution, (all solid driven in solution) aliquots were extracted and analyzed to ensure OPA concentration of 0.55%. The data reported below shows the dissolution characteristics of several formulations. Solids that dissolved in 1.5 h to 3.0 h are designated with one star (*). Solids that dissolved in 50 min to 1.5 h are designated with two stars (**). Solids that dissolved in 30 min to 50 min are designated with three stars (***) Finally, solids that dissolved in less than 30 min are designated with four stars (***

### Example 1

In a 100 mL beaker were added 7.5 g polyethylene glycol (MW 3400, Aldrich). The polyethylene glycol was melted in a hot water bath, following which 1.1 g ortho-phthalic aldehyde (OPA, Aldrich) were added. The heating was continued with gentle stirring, until all the OPA melted. The melted mixture was poured in a crystallizing dish, which was allowed to cool down and solidify. The solid was removed and pulverized using a mortar and pestle.

Dissolution Test

To 50 g water were added with agitation 2.0 g of the above powder (0.25 g active OPA. Upon complete dissolution, judged by the disappearance of all solid particles, aliquots were extracted and analyzed by HPLC. It was determined that the solution had the targeted OPA concentration of 0.55% OPA

Control I.: 0.25 g OPA were added with agitation to 50 g of H₂O. Upon complete dissolution, judged by the disappearance of all solid particles, aliquots were extracted and analyzed by HPLC. It was determined that the solution had the targeted OPA concentration of 0.55% OPA

Control II.: 1.75 g polyethylene glycol were dissolved in 50 g H₂O. Upon complete dissolution, 0.25 g OPA were added with agitation. Upon complete dissolution of OPA, judged by the disappearance of all solid particles, aliquots were extracted and analyzed by HPLC. It was determined that the solution had the targeted OPA concentration of 0.55% OPA

### Example 2

TABLE I
Water soluble polymers - OPA formulations.

| | | | | Dissolution |
|---|---|---|---|---|
| # | Polymer | % OPA^{a} | Type of formulation | time |
| 1 | OPA control | 100 | granular | ** |
| 2 | OPA control | 100 | Melt/block | * |
| 3 | PEG MW 3400 | 12.4 | Melt mix/flakes | **** |
| 4 | PEG MW 3400 Control | 12.4 | Physical mix | *** |
| 5 | PEG MW 3400 Control | 12.4 | OPA added to H₂O | *** |
| | | | in which PEG was | |
| | | | previously dissolved | |
| 6 | PEG MW 4000 | 25 | Melt mix/flakes | **** |
| 7 | PEG MW 4000 | 50 | Melt mix/flakes | *** |
| 8 | PEG MW 4000 | 66.7 | Melt mix/flakes | *** |
| 9 | Polyacrylic acid (PAA) MW 2000 | 25 | Melt mix/block | **** |
| 10 | Polyacrylic acid (PAA) MW 2000 | 25 | Physical mix | *** |
| | Control | | | |
| 11 | Polyaspartic acid | 25 | Melt mix/block | *** |
| 12 | Polyvinyl alcohol MW ≦ 13,000 | 25 | Melt mix/block | **** |
| 13 | Polyvinyl alcohol MW 13,000- | 25 | Melt mix/block | **** |
| | 26,000 | | | |
| 14 | Polyvinyl alcohol MW 30,000- | 25 | Melt mix/block | **** |
| | 50,000 | | | |
| 15 | Soluble starch DP˜7 | 25 | Melt mix/block | **** |
| 16 | Sugar | 25 | Melt mix/block | **** |
| 17 | Polyacrylamide MW 10,000 | 25 | Melt mix/block | **** |

^{a}OPA content of the solid formulation.

### Example 3

TABLE II
Carboxylic acid - OPA formulations.

| # | Carboxylic acid | % OPA^{a} | Type of formulation | Dissolution time |
|---|---|---|---|---|
| 1 | OPA control | 100 | granular | ** |
| 2 | OPA control | 100 | Melt/block | * |
| 3 | Citric acid | 25 | Melt/block | **** |
| 4 | Citric acid | 25 | Physical mix | ** |
| | | | Control | |
| 5 | Tartaric acid | 25 | Melt/block | **** |
| 6 | Glutaric acid | 25 | Melt/powder | **** |
| 7 | Succinic acid | 25 | Melt/powder | **** |
| 8 | Oxalic acid | 25 | Melt/powder | **** |

^{a}OPA content of the solid formulation.

### Example 4

TABLE III
Carboxylic acid salts - OPA fonnulations. The dissolution times reported
are for the formulations in the block form.

| | | | Dissolution |
|---|---|---|---|
| # | Salt | % OPA^{a} | time |
| 2 | OPA control | 100 | * |
| 3 | Sodium lactate | 25 | *** |
| 4 | Sodium L-tartrate dihydrate | 25 | **** |
| 5 | Potassium citrate | 25 | **** |
| | monohydrate | | |
| 6 | Potassium acetate | 25 | **** |
| 7 | Potassium acetate | 25 | **** |
| 8 | Sodium acetate | 25 | **** |

^{a}OPA content of the solid formulation.

### Example 5

TABLE IV
Salts - OPA formulations. The dissolution times reported are for the
formulations in the block form.

| | | | Dissolution |
|---|---|---|---|
| # | Salt | % OPA^{a} | time |
| 2 | OPA control | 100 | * |
| 3 | NaCl | 25 | *** |
| 4 | NaHCO₃ | 25 | ***** |
| 5 | NaHCO₃ + citric acid | 25 | *** |
| 6 | NaH₂PO₄ | 25 | **** |
| 7 | NaH₂PO₄ finely ground | 25 | *** |
| 8 | Na₂HPO₄ | 25 | *** |
| 9 | Na₃PO₄ | 25 | **** |
| 10 | (NaPO3)n | 25 | *** |

^{a}OPA content of the solid formulation.

### Example 6

TABLE V
Anionic surfactants - OPA formulations.

| # | Surfactant | % OPA^{a} | Type of formulation | Dissolution time |
|---|---|---|---|---|
| 1 | OPA control | 100 | granular | ** |
| 2 | OPA control | 100 | Melt/block | * |
| 3 | Sodium dodecyl sulfate | 25 | Melt/block | **** |
| 4 | Sodium dodecyl sulfate | 25 | Physical mix | *** |
| 5 | HAMPOSYL L-95 | 25 | Melt/block | **** |
| 6 | HAMPOSYL L-95 | 25 | Physical mix | *** |

^{a}OPA content of the solid formulation.

### Example 7

TABLE VI
Three-Component Systems. In all cases, the formulations
contained 25% OPA.

| | | | | |
|---|---|---|---|---|
| | Weight | | Weight | Dissolution |
| Component A | % A | Component B | % B | time |

Effect of disintegrating agents on PEG based formulations

| | | | | |
|---|---|---|---|---|
| PEG 4000 | 37.5 | Potassium | 37.5 | **** |
| | | acetate | | |
| PEG 4000 | 37.5 | Sodium acetate | 37.5 | **** |
| PEG 4000 | 37.5 | Sodium acetate | 37.5 | **** |
| | | trihydrate | | |

Effect of surfactants on PEG formulations

| | | | | |
|---|---|---|---|---|
| PEG 4000 | 37.5 | HAMPOSYL^{c} | 37.5 | **** |
| | | L95 | | |
| PEG 4000 | 73.1 | Dowfax2A1- | 1.9 | **** |
| | | D | | |
| PEG 4000 | 72.3 | TERGITOL | 2.7 | **** |
| | | 15-S-40 | | |
| PEG 4000 | 75 | | | **** |

Miscellaneous mixes

| | | | | |
|---|---|---|---|---|
| PEG 4000 | 37.5 | Glutaric acid | 37.5 | **** |
| Potassium | 37.5 | Citric acid | 37.5 | **** |
| citrate | | | | |

^{a}HAMPOSYL L-95 is the tradename for sodium lauroyl sarcosinate supplied by the Dow Chemical Company.
^{b}DOWFAX 2A1-D is the Dow tradename for 1,1-oxybistetrapropylene diphenyl disulfonate sodium salt
^{c}TERGITOL 15-S-40 is the Dow tradename for alkyloxypolyethyleneoxyethanol.

### Example 8

Two fifty-mL aliquots of Tryptic Soy Broth was inoculated with Pseudomonas aeruginosa ATCC #10145 and Staphylococcus aureus ATCC #6538, respectively, and incubated overnight at 37° C. The cultures were then pelleted by centrifugation at 4000 rpm for 20 minutes, decanted, resuspended in sterile phosphate buffered water and re-pelleted to wash. The cells were then resuspended to a 1×10⁸ CFU/mL concentration. Seven different liquid formulations of OPA were prepared at 50-ppm concentration and inoculated with the bacteria. Initial bacterial concentrations in the diluted OPA formulations were 1×10⁶ and 1×10⁷ CFU/mL for the S. aureus and P. aeruginosa, respectively. Each inoculated formulation was then plated after 30 minutes of contact time, to determine the total number of viable organisms in colony forming units per milliliter (CFU/mL) on Tryptic Soy Agar. The formulations were as follows:

Formulations of OPA tested for microbiological efficiency

| | |
|---|---|
| F1 | 3.0667 g of solid OPA |
| | 9.0115 g PEG 8000 = |
| | 25.39% OPA |
| F2 | 3.0061 g of solid OPA |
| | 8.5890 g of PEG 8000 |
| | 0.5946 g of EDTA = |
| | 24.66% OPA |
| F3 | 3.0048 g of solid OPA |
| | 8.7218 g of PEG 8000 |
| | 0.2615 g of HAMPOSYL L95 = |
| | 25.06% OPA |
| F4 | 3.0373 g of solid OPA |
| | 4.5 800 g of PEG 8000 |
| | 4.5089 g of potassium acetate = |
| | 25.05% OPA |
| F5 | 3.1839 g of solid OPA |
| | 9.0368 g of glutaric acid = |
| | 26.05% OPA |
| F6 | 0.4917 g of solid OPA in 9.000 g water (50 ppm) |
| F7 | Cidex OPA, 0.55% OPA |

TABLE VIII
The efficiency of various OPA formulations (at 50 ppm OPA
concentration) against Staphylococcus aureus (ATCC #6538).
Results are reported as surviving numbers of bacteria (CFU/mL)

Contact time

| Formulation | 0 min | 30 min |
|---|---|---|
| F1 | 1 × 10⁶ | 0 |
| F2 | 1 × 10⁶ | 0 |
| F3 | 1 × 10⁶ | 0 |
| F4 | 1 × 10⁶ | 0 |
| F5 | 1 × 10⁶ | 0 |
| F6 | 1 × 10⁶ | 0 |
| F7 | 1 × 10⁶ | 0 |
| Control | 1 × 10⁶ | 1 × 10⁶ |

TABLE IX
The efficiency of various OPA formulations (at 50 ppm OPA
concentration) against Pseudomonas aeruginosa (ATCC #10145).
Results are reported as surviving numbers of bacteria (CFU/mL)

Contact time

| Formulation | 0 min | 30 min |
|---|---|---|
| F1 | 1 × 10⁷ | 20 |
| F2 | 1 × 10⁷ | 3 |
| F3 | 1 × 10⁷ | 40 |
| F4 | 1 × 10⁷ | 100 |
| F5 | 1 × 10⁷ | 0 |
| F6 | 1 × 10⁷ | 1000 |
| F7 | 1 × 10⁷ | 1000 |
| Control | 1 × 10⁷ | 1 × 10⁷ |

While the invention has been described herein, with respect to certain features, aspects and embodiments, it will be recognized that the invention may be widely varied, and that numerous other modifications, variations and other embodiments are possible, and that such modifications, variations and embodiments are to be regarded as being within the spirit and scope of the invention.

What is claimed is:

1. A solid formulation of Ortho-Phthalic aldehyde (OPA) mixed with one or more compositions selected from the group consisting of water soluble polymers, carboxylic acids, surfactants, and salts wherein the formulation contains from 0.1% to 99.9% OPA, better 10% to 50% OPA, best about 20 to 30% OPA.

2. The formulation of claim 1 wherein the water soluble polymer is selected from the group consisting of polyethylene glycol, starch, soluble cellulose based polymers, polyvinyl alcohol, polyacrylic acid, polyacrylates, polyacrylamides, sugar, polymeric carbohydrates, and polyaspartic acid.

3. The formulation of claim 1 wherein the surfactant is selected from the group of anionic, cationic, amphoteric, or nonionic surfactant.

4. The formulation of claim 3 where the anionic surfactant is selected from the group of sodium dodecyl sulfate, 1,1-oxybistetrapropylene diphenyl disulfonate sodium salt, dinonyl sulfoccinate, and sodium lauroyl sarcosinate,

5. The formulation of claim 3 where the cationic surfactant is selected from the group consisting of quaternary ammonium salts and alkyl pyridinium salts.

6. The formulation of claim 3 where the amphoteric surfactant is selected from the group consisting of betaines and imidazoline amphoterics.

7. The formulation of claim 3 where the nonionic surfactant is selected from the group consisting of alkoxylates, ethylene oxide/propylene oxide block copolymers, amine oxides, and lauroyl sarcosine.

8. The formulation of claim 1, wherein the carboxylic acid is selected from the group consisting of citric acid, tartaric acid, glutaric acid, malonic acid, adipic acid, succinic acid, and oxalic acid.

9. The formulation of claim 1 wherein the salt is a carboxylic acid salt such as, but not limited to sodium lactate, sodium L-tartrate dihydrate, potassium citrate monohydrate, sodium acetate, sodium acetate trihydrate, and potassium acetate.

10. The formulation of claim 1 where the salt is selected from the group consisting of sodium chloride, sodium bicarbonate, NaH₂PO₄, Na₃PO₄ and (NaPO₃)n.

11. The formulation of claim 1 further comprising a disintegrating agent.

12. The formulation of claim 11 wherein the disintegrating agent is selected from the group of potassium acetate, sodium acetate, sodium acetate trihydrate, and Acusol® polymers.

13. The formulation of claim 1 further comprising a chelating agent.

14. The formulation of claim 13 wherein the chelating agent is selected from the group consisting of ethylenediamine N,N,N′,N′ tetraacetic acid, sodium salts of ethylenediamine N,N,N′,N′ tetraacetic acid, hexamethylenediamine N,N,N′,N′ tetraacetic acid, nitrilotriacetic acid, and citric acid.

15. The formulation of claim 1 further comprising a stabilizer.

16. The formulation of claim 15 wherein the stabilizer is selected from the group consisting of water miscible alcohols, dicarboxylic acid esters, propylene carbonate, N,N-dimethylacetamide and butyrolactone.

17. A disinfection method comprising treating one or more objects with a disinfectant solution comprised of a solid formulation OPA mixed with one or more compounds selected from the group consisting of water soluble polymers, carboxylic acids, surfactants, and salts wherein the formulation contains from 0.1% to 99.9% OPA, better 10% to 50% OPA, best about 20 to 30% OPA dissolved in a solvent selected from the group consisting of water and mixtures of water with water-miscible-solvents.

18. The method of claim 17 wherein the solid formulation of OPA is in the form of a treatment applicator selected from the group consisting of a tablet, a block, a pellet, a powder and a puck.

19. The method of claim 17 wherein the disinfectant solution further comprises a disintegrating agent such as but not limited to potassium acetate, sodium acetate, sodium acetate trihydrate.

20. The method of claim 17 wherein the treated object is one used in an application selected from the group consisting of instrument sterilization; household an industrial cleaning, sanitizing and disinfecting; industrial water treatment including cooling water, pulp and paper and oil field treatment; drilling mud preservation; sump treatment for metal working systems; a solid disinfectant for toilet bowls and porta-potties; treatment of aqueous paints, adhesives, latex emulsions, and joint cements; preserving wood; preserving cutting oils; a spray or dip treatment for textiles and leather; as a component of anti-fouling paints; protecting paint films; protecting cane and beet sugar processing equipment; treatment of air washer or scrubber systems and in industrial fresh water supply systems; treatment of various specialty boards, including cardboard and particle board; preventing sap stain discoloration on freshly cut wood; treatment of clay and pigment slurries; as a hard surface disinfectant; and as a swimming pool treatment.

21. The method of claim 17 wherein the treated object is one used in an application selected from the group consisting of the paint, wood, textile, cosmetic, leather, tobacco, fur, rope, paper, pulp, plastics, fuel, oil, rubber, and machine industries, household an industrial applications.

22. A process for creating a fast-dissolving OPA mixture comprising the steps of:
a) melt mixing OPA with one or more compounds selected from the group consisting of water soluble polymers, carboxylic acids, surfactants, and salts to create a melt mixture:
b) cooling the melt mixture; and
c) pulverizing the cooled melt mixture,
wherein the cooled melt mixture contains from 0.1% to 99.9% OPA, better 10% to 50% OPA, best about 20 to 30% OPA.

23. The process of claim 22 wherein the step of pulverizing comprises a process selected from the group consisting of drum flaking, conveyor flaking, pastillization, prilling, freeze spraying, jet precipitation or solidification, extrusion into strands or films, and pulverization/grinding/chopping.

24. The formulation of claim 1 further comprising a flow agent selected from the group consisting of inorganic salts, organic salts, fumed silica and precipitated silica.

25. The process of claim 22 wherein the step of melt mixing comprises melting the additive(s) selected from the group consisting of water soluble polymers, surfactants, carboxylic acids, carboxylic acid salts, and salts in a hot water bath at a temperature of about 100° C. followed by addition of ortho-phthalic aldehyde.

26. The process of claim 22 wherein the step of melt mixing comprises melting the ortho-phthalic aldehyde in a hot water bath at a temperature of 80-100° C. followed by addition of one or more additives selected from the group consisting of water soluble polymers, surfactants, carboxylic acids, and salts and mixing to ensure good dispersion of the unmelted additive(s) in the molten OPA.

27. The processes of claim 22 wherein the ratio of ortho-phthalic aldehyde to the second composition is from 0.1% to 99.9% OPA, better 10% to 50% OPA, best about 20 to 30% OPA.

28. The process of claims 25 and 26 wherein the step of melt mixing further comprises gentle stirring until the OPA is completely melted.

29. The processes of claims 25 and 26 wherein the step of melt mixing further comprises adding one or more compositions selected from the group consisting of surfactant, chelating agent, disintegrating agent, buffering salts, antiscaling agent, organic matter dispersant, and other biocides.

30. The process of claim 22 wherein the step of melt mixing comprises substituting co-extrusion for melt mixing.
